(19) United States
(12) Patent Application Publication
(10) Pub. No.: US 2021/0030285 A1
(43) Pub. Date: Feb. 4, 2021

(54) BIOLOGICAL INFORMATION DETECTION DEVICE

(71) Applicant: HITACHI, LTD., Tokyo (JP)

(72) Inventors: Nobuhiro FUKUDA, Tokyo (JP); Hironori WAKANA, Tokyo (JP); Takashi NUMATA, Tokyo (JP)

(73) Assignee: HITACHI, LTD., Tokyo (JP)

(21) Appl. No.: 16/942,862

(22) Filed: Jul. 30, 2020

(30) Foreign Application Priority Data
Aug. 2, 2019 (JP) 2019-142775

(51) Int. Cl. A61B 5/021; A61B 5/026; A61B 5/0285; A61B 5/103; G06T 5/00

(52) U.S. Cl. CPC A61B 5/02108; A61B 5/0261; A61B 5/02427; A61B 5/1032; G06T 5/001; A61B 5/0285

(57) ABSTRACT

A biological information detection device is robust to color change of external light or illumination light. The biological information detection device includes: an image acquiring section that acquires image information by taking an image of the face of a living body; a blood flow analyzing section that corrects the image information according to the Retinex theory for color constancy, outputs hue information in the corrected image information as blood flow information, and outputs skin area mark information indicating the position of a given skin area in the face; and a local pulse wave detecting section that obtains, from the blood flow information of the skin area corresponding to the skin area mark information, pulse information of the skin area.

### TECHNICAL FIELD

The present invention relates to a biological information detection device that detects the biological information of a living body in a noncontact manner in real time.

### BACKGROUND

Techniques that acquire biological information in a noncontact manner in real time using microwaves or a camera are available. Particularly, in the pulse acquisition techniques which use a camera, the tendency toward smaller camera modules is growing and the use of camera modules mounted in mobile terminals including smartphones is spreading.

In addition, by application of pulse detection in image information, a stress index which represents a balance of autonomic nerves can be obtained, for example, by monitoring the pulse interval, namely R-wave interval (RRI). Furthermore, research has been promoted to develop the techniques to monitor various kinds of biological information for elderly households or for detection of a sudden change in the health condition of a person who is driving a car.

For example, Japanese Patent Application Publication No. 2018-086130 describes a pulse detection method which is hardly affected by change in the imaging environment by measuring the change in the wavelength distribution of an image of blood flows.

Specifically, in Japanese Patent Application Publication No. 2018-086130, paying attention to the fact that the amount of change is different among the signal components of an RGB signal in a face image, a color component of the face image is separated into the wavelength of reflected light and spectral intensity and particularly the change in wavelength distribution which is not affected by the change in external light luminance or spectral intensity is measured. Since the change in wavelength distribution is correlated with hue change in the color space, pulse detection less susceptible to the change in the luminance of external light can be made by measuring the temporal change in hue.

### SUMMARY OF THE INVENTION

When the technique described in Japanese Patent Application Publication No. 2018-086130 is applied to a driver monitoring device, one problem is that while the car is passing a building or under the shade of a tree or the like, though the device can cope with the change in the brightness of external light, it cannot avoid the influence of illumination light with a wavelength distribution different from that of external environmental light, such as a neon or other type of lamp.

An object of the present invention is to provide a biological information detection device that is robust to color change of external light or illumination light.

In order to solve the above problem, according to one aspect of the present invention, there is provided a biological information detection device that includes: an image acquiring section that acquires image information by taking an image of the face of a living body; a blood flow analyzing section that corrects the image information according to the Retinex theory for color constancy, takes hue information in the corrected image information as blood flow information, and outputs skin area mark information indicating the position of a given skin area in the face; and a local pulse wave detecting section that obtains, from the blood flow information in the skin area corresponding to the skin area mark information, pulse information of the skin area.

According to the present invention, there is provided a biological information detection device that is robust to color change of external light or illumination light.

### BRIEF DESCRIPTION OF DRAWINGS

FIG. 1 is a block diagram which shows the general structure of a biological information detection device,

FIG. 2 is a view which explains the blood flows in the face,

FIG. 3A shows a frame image which contains the skin areas in which images of the blood flows in the forehead, right buccal surface, and left buccal surface for pulse wave detection are acquired,

FIG. 3B shows an example of pulse information 51 (pulse wave information),

FIG. 4 is a processing flowchart which summarizes the biological information detection device,

FIG. 5 is a block diagram which shows the structure of the blood flow analyzing section,

FIG. 6 is a diagram which explains the detailed structure of the image correcting section,

FIG. 7 is a structure diagram of the local pulse wave detecting section,

FIG. 8 is a flowchart which explains the sequence in which the pulse wave velocity calculating section acquires the pulse wave velocity,

FIG. 9 is a block diagram which explains another feature of the blood flow analyzing section,

FIG. 10 is a structure diagram of the image correcting section which receives face area mark information, and

FIG. 11 is a diagram which explains another feature of the image correcting section.

### DETAILED DESCRIPTION

Next, an embodiment of the present invention will be described in detail referring to drawings.

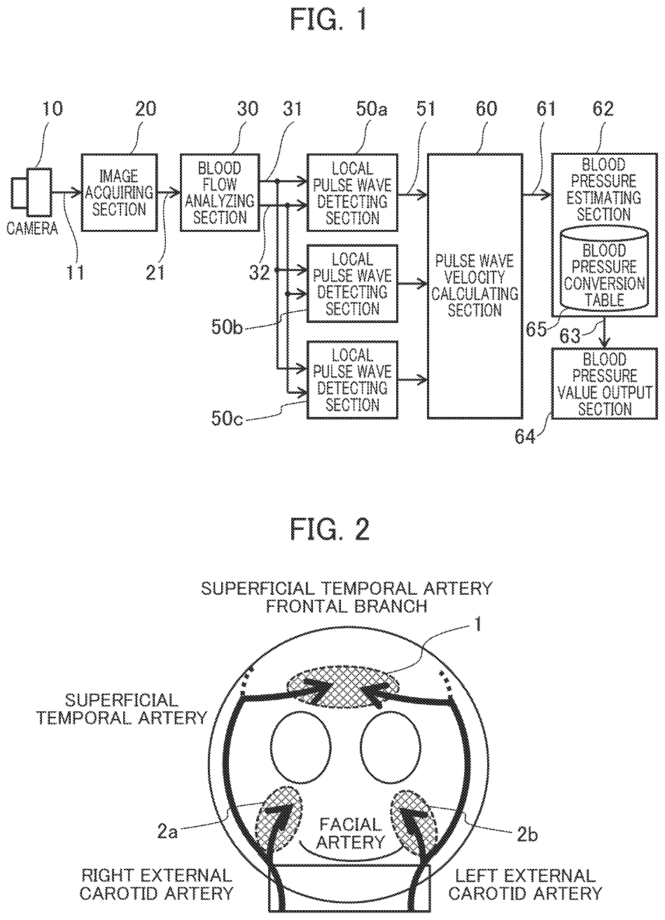

FIG. 1 is a block diagram which shows the general structure of a biological information detection device according to the embodiment.

The biological information detection device according to the embodiment takes advantage of the characteristic of hemoglobin in the blood that it easily absorbs green light. The device takes an image of reflected light of the light irradiated on a living body, analyzes the blood flow and calculates the pulse/blood pressure according to the change in the spectral distribution of the reflected light.

The biological information detection device in FIG. 1 includes a camera 10, an image acquiring section 20, a blood flow analyzing section 30, three local pulse wave detecting sections 50a, 50b, and 50c (hereinafter these sections may be collectively designated as 50), a pulse wave velocity calculating section 60, a blood pressure estimating section 62, and a blood pressure value output section 64.

The image acquiring section 20 acquires an image signal 11 from the camera 10 as imaging information of reflected light from the living body at a prescribed frame rate and converts the imaging information into image data 21 in the RGB color system and outputs the data in a time-series manner for later analysis. Alternatively, the image acquiring section 20 may acquire the imaging information of reflected light from the living body through a signal cable or communication network or through a storage device such as an image recorder, instead of through the image signal 11 from the camera 10.

As will be detailed later, the biological information detection device analyzes the blood flow according to the change in reflected light between frames of the imaging information acquired from the camera 10.

The blood flow analyzing section 30 analyzes the received image data 21 in each frame, extracts an image area including a blood flow image (hereinafter called a skin area) and outputs blood flow information 32 including blood reflected light information and skin area mark information 31 for acquisition of a blood flow image for each frame.

The local pulse wave detecting sections 50a, 50b, and 50c, each provided fora skin area including a blood flow image, detects the pulse wave of the blood flow (blood vessel) from the time-series change in the blood flow reflected light value according to the reflected light value of the blood flow in the blood flow information 32 analyzed by the blood flow analyzing section 30 and received frame by frame, adds the detected pulse wave change to the blood flow information 32 and outputs it as pulse wave information 51.

Specifically, the volumetric change of the blood vessel as caused by the blood flow change synchronized with the pulsation of the heart is detected as change in the spectral distribution of the blood flow reflected light and the temporal change in the spectral distribution is taken as a pulse wave.

The pulse wave velocity calculating section 60 calculates the pulse wave velocity (PWV) 61 according to a plurality of pieces of pulse wave information 51 detected by the local pulse wave detecting sections 50a, 50b, and 50c. Specifically, the velocity is calculated by dividing the difference in the distance from the heart between the pulse wave detecting areas by the pulse wave phase difference.

The blood estimating section 62 estimates blood pressure information 63 from the pulse wave velocity 61 according to the Moens-Korteweg blood vessel model and the relation between blood vessel wall elasticity and blood pressure.

The blood pressure value output section 64 outputs the blood pressure information 63 estimated by the blood pressure estimating section 62 to a display unit or terminal.

The blood pressure conversion table 65 is a storage area for a table showing the correspondence relation between pulse wave velocity 61 and blood pressure information 63.

The functions of the above various sections which constitute the biological information detection device can be implemented by hardware circuitry which uses a special integrated circuit (FPGA: Field Programmable Logic Array, etc.), except the camera 10. Alternatively, the functions can be implemented by a computer including a processor, a storage unit (semiconductor memory, hard disk unit, etc.), an input/output device (communication device, keyboard, mouse, display unit, etc.). In this case, the functions of the various sections which constitute the biological information detection device are performed by the processor which executes the program stored in the storage unit.

Specifically, the computer as the biological information detection device receives the image data 21 through the input/output device and the processor performs the functions as the blood flow analyzing section 30, local pulse wave detecting sections 50, pulse wave velocity calculating section 60, and blood pressure estimating section 62 according to the program, and the input/output device outputs blood pressure information.

Next, the functions of the biological information detection device according to the embodiment will be summarized referring to FIG. 2 to FIG. 4.

FIG. 2 is a view which explains the blood flows in the face whose image is to be taken by the camera 10.

It is known that in the head of a living body, the blood circulates from the heart to the face and scalp through the “left external carotid artery” branched from the “left common carotid artery” and the “right external carotid artery” branched from the “right common carotid artery”. As shown in FIG. 2, the blood is transported to the right buccal surface 2a of the face through the “facial artery” branched from the “right external carotid artery” and the blood is transported to the left buccal surface 2b of the face through the “facial artery” branched from the “left external carotid artery”. The blood is transported to the forehead 1 through the “superficial temporal artery frontal branch”. The “superficial temporal artery frontal branch” is a branch of the “superficial temporal artery” as one of the terminal branches of the “right external carotid artery” and “left external carotid artery”.

As mentioned above, the forehead 1 is located in a remoter place from the heart than the right buccal surface 2a and left buccal surface 2b and is suppled with blood through different blood vessels, so the pulse waves in the right buccal surface 2a and left buccal surface 2b are different in phase from the pulse wave in the forehead 1. Specifically, the phase of the pulse wave in the forehead 1 is later than the phases of the pulse waves in the right buccal surface 2a and left buccal surface 2b.

More specifically, since the path from the heart to the “right common carotid artery” and the path to the “left common carotid artery” are different, a phase difference occurs even between the pulse wave in the right buccal surface 2a and the pulse wave in the left buccal surface 2b. If this phase difference is not more than a prescribed value, it can be determined that normal pulse waves in the right buccal surface 2a and left buccal surface 2b have been detected.

In the biological information detection device according to the embodiment, three blood flows in the skin areas of the forehead 1, right buccal surface 2a, and left buccal surface 2b of the face are detected. However, in estimating the blood pressure by calculating the pulse wave velocity from the pulse information (pulse wave information), the blood pressure can be estimated from only two pieces of pulse information. In other words, the blood pressure can be estimated from the pulse information of the forehead 1 and the pulse information of the right buccal surface 2a or left buccal surface 2b.

Therefore, in the biological information detection device according to the embodiment, the blood pressure is estimated either from the pulse information of the forehead 1 and that of the right buccal surface 2a or from the pulse information of the forehead 1 and that of the left buccal surface 2b. This increases the tolerance in the face imaging direction and reduces the restriction on the orientation of the face, thereby leading to improvement in the convenience and accuracy of the biological information detection device.

Whether to select the pulse information of the right buccal surface 2a or the pulse information of the left buccal surface 2b as pulse information is determined according to the appropriateness as pulse information. If the pulse information of the right buccal surface 2a and the pulse information of the left buccal surface 2b are both appropriate, the average information is adopted.

The blood is also transported to the face not only through the “facial artery” and the “superficial temporal artery” but also through other arteries. For this reason, in the whole face the distance from the heart differs from one area to another and thus a pulse wave (pulse) phase difference occurs between areas. In the biological information detection device according to the embodiment, the pulse waves in the skin areas of the forehead 1, right buccal surface 2a, and left buccal surface 2b of the face are detected, though not limited to these areas.

As mentioned above, the biological information detection device according to the embodiment detects the blood flows in at least three skin areas in which the blood flows have a phase difference. Specifically, the device detects the blood flow in one skin area which lies on the centerline of the face and the blood flows in the other skin areas which lie symmetrically with respect to the centerline of the face and are shorter in blood flow path length to the heart than the skin area on the centerline. This increases the tolerance in the face imaging direction and reduces the restriction on the orientation of the face, thereby leading to improvement in the convenience and accuracy of the biological information detection device.

Next, division into areas for the forehead 1, right buccal surface 2a, and left buccal surface 2b, in which pulse waves (pulses) are detected, and detection of pulse wave phase difference will be explained.

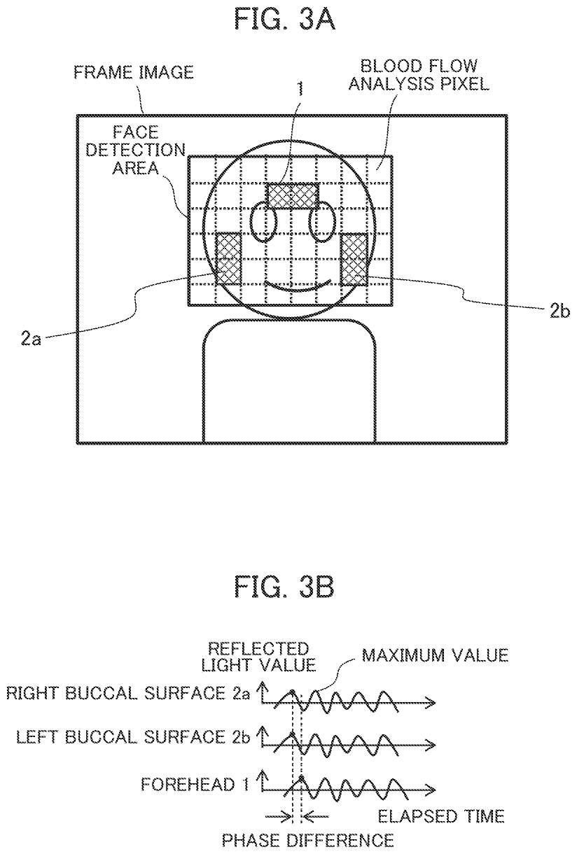

FIG. 3A shows a frame image which contains the skin areas in which images of the blood flows in the forehead 1, right buccal surface 2a, and left buccal surface 2b for detection of pulse waves are acquired, in the imaging information of the reflected light from the living body imaged by the camera 10. The imaging information is information on frame images arranged in a time-series manner, with pixels arranged two-dimensionally in each frame.

The biological information detection device extracts the face from each frame image in the imaging information using the Viola-Jones algorithm or the like and extracts the pixels corresponding to the skin areas of the forehead 1, right buccal surface 2a, and left buccal surface 2b from the image area in which the face has been detected (face detection area). Then, for each extracted skin area, the spectral distribution values of blood flow reflected light as indicated by the pixels are added together or averaged to obtain blood flow information 32.

The biological information detection device arranges the blood flow information 32 in each of the skin areas in a time-series manner and takes it as pulse wave information.

FIG. 3B shows an example of pulse information 51 (pulse wave information).

In a skin area of a living body, with the blood vessel volumetric change caused by blood flow change, the amount of hemoglobin in the skin area increases or decreases, which results in a change in the spectral distribution value of reflected light. Therefore, by arranging the reflected light values in the blood flow information 32 in a time-series manner, as shown in FIG. 3B, the pulse waveform (pulse information) which corresponds to the heartbeat cycle can be obtained for each of the right buccal surface 2a, left buccal surface 2b, and forehead 1.

As will be detailed later, in the biological information detection device, the phase difference between skin areas is detected by obtaining the pulse waves from the temporal change in the spectral distribution value (hue) of reflected light. For the purpose of explanation, FIG. 3B shows the pulse waves according to the temporal change in the reflected light value, in which the phase difference between skin areas is the same (also the same in the subsequent figures).

The pulse wave phase differences of the right buccal surface 2a, left buccal surface 2b, and forehead 1 can be obtained by calculating the time difference of the maximum value or minimum value of each pulse waveform as shown in FIG. 3B.

As mentioned above, since the pulse wave of the forehead 1 is later than the pulse wave of the right buccal surface 2a or left buccal surface 2b, the blood pressure can be estimated by calculating the pulse wave velocity from the obtained phase difference.

As will be detailed later, the biological information detection device specifies or judges the skin area for pulse wave detection as follows to obtain the blood flow information 32.

One method is to register the color of the forehead 1, right buccal surface 2a, and left buccal surface 2b of the face of the living body (subject) for pulse wave detection, as skin area judgement color and make reference to it to obtain the blood flow information 32. Specifically, as color information in the imaging information, the range of skin area judgement color is defined and if the color of pixels in the frame image is the judgement color, the pixels are taken as skin area pixels and used to obtain the blood flow information 32.

Another method is to register the area coordinates (pixel position information) of the skin areas of the forehead 1, right buccal surface 2a, and left buccal surface 2b and extract pixels from the frame image according to the area coordinates of the skin areas to obtain the blood flow information 32 as skin area pixels.

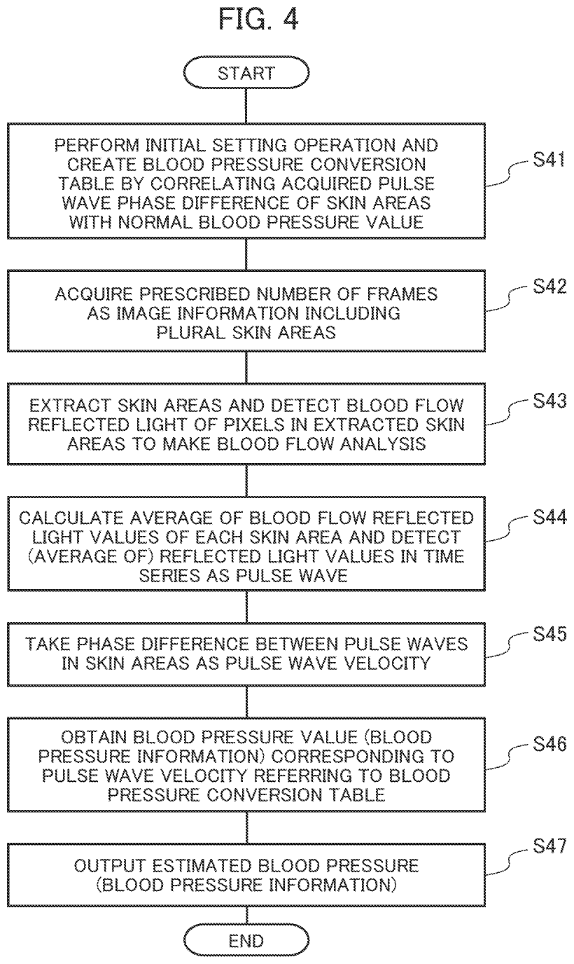

Next, operation of the biological information detection device will be summarized referring to FIG. 4.

In the processing flow in FIG. 4, the blood pressure is estimated by a method in which reference is made to the correspondence table (blood pressure conversion table 65) of pulse wave phase differences and blood pressure values, different from the method in which the blood pressure information 63 is estimated from the pulse wave velocity 61 according to the Moens-Korteweg blood vessel model and the relation between blood vessel wall elasticity and blood pressure.

At Step S41, as initial setting operation, the biological information detection device detects the pulsating flow information of the living body (subject) in his/her normal state for each skin area, calculates the pulse wave (pulse) phase difference and registers it in the blood pressure conversion table 65 and also registers the actual blood pressure value measured with a sphygmomanometer at this time in correlation with the phase difference to create a blood pressure conversion table 65.

It is preferable that sets of pulse wave phase difference and blood pressure value under different conditions should be registered in the blood pressure conversion table 65.

At Step S42, the image acquiring section 20 of the biological information detection device acquires a prescribed number of frames, each of which is the image information of reflected light from the face of the living body or the like.

At Step S43, for blood flow analysis, the blood flow analyzing section 30 of the biological information detection device extracts the face of the living body (subject) in each frame of the acquired image information, further extracts the skin areas of the forehead 1, right buccal surface 2a, and left buccal surface 2b from the extracted face image, and detects the pixel values of the skin areas as blood flow reflected light values to make blood flow analysis.

At Step S44, the local pulse wave detecting sections 50 (50a, 50b, 50c) of the biological information detection device calculate the average of the blood flow reflected light values in each of the skin areas extracted at Step S43. Then, the local pulse wave detecting sections 50 of the biological information detection device detect the average of blood flow reflected light values of frames (time-series) as pulse wave information (pulse wave) of each skin area.

At Step S45, the pulse wave velocity calculating section 60 evaluates the appropriateness of the pulse wave information of the skin areas of the right buccal surface 2a and left buccal surface 2b as detected at Step S44 and calculates the phase difference between the pulse wave in the skin area of the forehead 1 and the pulse wave in the right buccal surface 2a, the phase difference between the pulse wave in the skin area of the forehead 1 and the pulse wave in the left buccal surface 2b or the average of the two phase differences and takes this as the pulse wave velocity value.

At Step S46, the blood pressure estimating section 62 of the biological information detection device obtains the blood pressure value corresponding to the pulse wave velocity (phase difference) calculated at Step S45 in reference to the blood pressure conversion table 65 registered at Step S41 and takes it as estimated blood pressure (blood pressure information).

At Step S47, the blood pressure value output section 64 outputs the blood pressure information obtained at Step S46 to the display unit or terminal.

Next, the various blocks of the biological information detection device shown in FIG. 1 will be explained in detail.

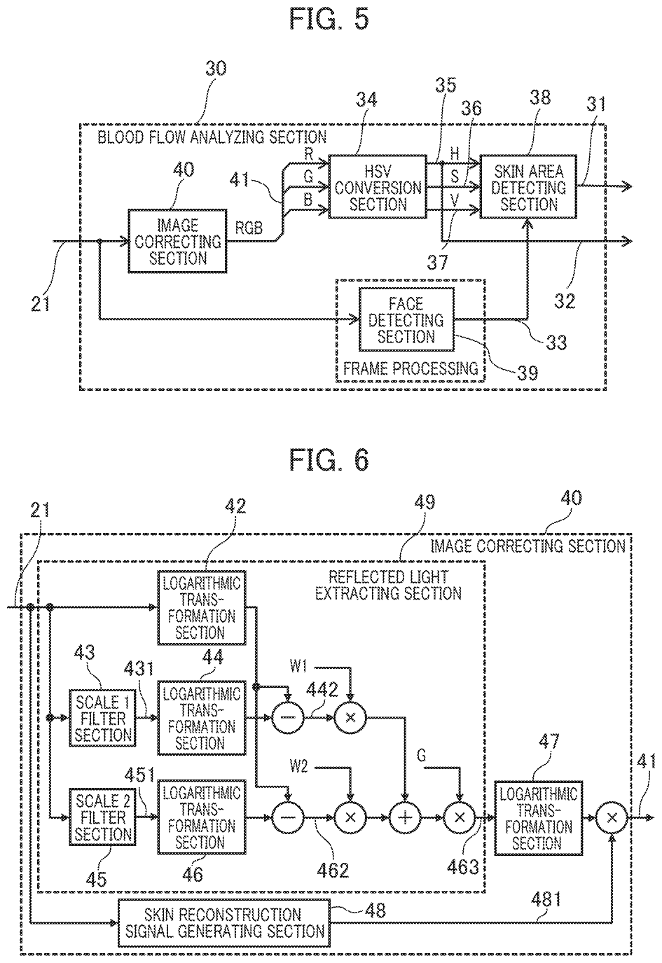

FIG. 5 is a block diagram which shows the structure of the blood flow analyzing section 30. The blood flow analyzing section 30 includes an image correcting section 40, an HSV conversion section 34, a skin area detecting section 38, and a face detecting section 39 and performs image processing of each pixel in the image data 21.

As will be detailed later, the image correcting section 40 is a processing section which receives the image data 21 and eliminates the influence of the illumination light component in the image data 21 by image correction processing based on the Retinex theory.

The HSV conversion section 34 receives the unpacked image information 41 as the result of separation of the image data corrected by the image correcting section 40 into R (red), G (green), and B (blue) image data, and converts this into image data in the color system of the HSV color space which includes hue information 35 (H), saturation information 36 (S), and brightness value information 37 (V).

In the biological information detection device, blood flow change is taken as change in the amount of blood hemoglobin per unit area and the change in the spectral distribution of reflected light as the result of absorption of green light by hemoglobin is detected. In order to facilitate this detection process, the HSV conversion section 34 converts the image data in the RGB color system into image data in the HSV color system to perform the blood flow detection process. Consequently, the hue information 35 (H) is outputted as the blood flow information 32 which is output information from the blood flow analyzing section 30.

The face detecting section 39 receives the image data 21, detects the face in each frame, for example, by the Viola-Jones method and outputs the face area mark information 33 indicating the position of the face area including the skin area for blood flow detection, to the skin area detecting section 38.

The face detecting section 39 enables simultaneous detection or selective detection of blood flows in a plurality of living bodies (subjects), though not explained in detail here.

The skin area detecting section 38 receives the hue information 35 (H), saturation information 36 (S), and brightness value information 37 (V), and the face area mark information 33 and outputs the skin area mark information 31 which indicates the inclusion of a blood flow image.

The skin area detecting section 38 is explained in detail below.

The skin area detecting section 38 adopts one of the following methods: one method in which the color space range of the skin area (partial color space) is specified and if the color space of pixels in the image data as the result of conversion of the image data 21 into data in the HSV color system is in the color space range of the skin area, the skin area mark information 31 is outputted (first skin area detecting method) and the other method in which the area position of the skin area is specified and if the pixels in the image data as the result of conversion of the image data 21 into data in the HSV color system are in the specified area position range, the skin area mark information 31 is outputted (second skin area detecting method).

More specifically, in the first skin area detecting method, the color space range of the skin areas of the forehead 1, right buccal surface 2a, and left buccal surface 2b as illustrated in FIG. 3A is specified to output the skin area mark information 31. In the second skin area detecting method, the pixel positions of the areas of the forehead 1, right buccal surface 2a, and left buccal surface 2b are specified to output the skin area mark information 31.

Next, the structure of the image correcting section 40 will be explained in more detail.

The image correcting section 40 separates the illumination light component from an image according to the Retinex theory which suggests the human eye's visual sensation characteristics such as color constancy and brightness constancy to extract the reflected light component. This eliminates the influence of change in the wavelength distribution of external light or illumination light.

In the Retinex theory, many models which differ in the method of estimating the illumination light component or reflected light component are available. Among them, the Retinex model which extracts the reflected light component on the assumption that the local illumination light component follows the Gaussian distribution is called Center/Surround (hereinafter C/S) Retinex.

Representative Retinex models include Single Scale Retinex model (hereinafter SSR) and Multiscale Retinex model (hereinafter MSR). The image correcting section 40 adopts the MSR model.

According to the Retinex theory, an image I in a given pixel (x, y) is expressed by the product of illumination light L(x, y) and reflectance r (x, y) and thus can be described as I (x, y)=L (x, y)·r(x, y). Therefore, by estimating L (x, y), the image with reflectance r(x, y) can be reconstructed according to r(x, y)=I (x, y)/L(x, y).

In C/S Retinex, assuming that illumination light L follows the Gaussian distribution centered on the pixel concerned in the image, component R related to reflection in logarithmic space is calculated from the difference between the Gaussian distribution in the logarithmic space and the pixel concerned. The component R is expressed by Equation (1) below, in which I (x, y) denotes the luminance value of the pixel concerned and F(x, y) denotes gaussian:

Equation (1)

R(x,y)=log I(x,y)−log[F(x,y)⊗/(x,y)] (1)

In Equation (1), the Gaussian distribution with standard deviation σ, centered on the origin of a two-dimensional space, is expressed by Equation (2) below. (Here, the standard deviation represents the spread of the Gaussian distribution, so hereinafter it will be called “scale”.)

$$\begin{matrix}Equation ( 2 ) & \\ Gauss ( x , y , σ ) = \frac{1}{\sqrt{2 π} σ} {e}^{- \frac{{x}^{2} + {y}^{2}}{2 {σ}^{2}}} & ( 2 )\end{matrix}$$

The product of F(x, y) and I (x, y) in Equation (1) is called convolution product and expressed by Equation (3) below.

$$\begin{matrix} Equation ( 3 ) & \\ f ( x , y ) ⊗ g ( x , y ) ≡ {\int \int }_{Ω} f ( σ , τ ) g ( x - σ , y - τ ) d σ d τ \approx {{\sum }^{L}}_{s = - L} {{\sum }^{L}}_{t = - L} f ( s , t ) g ( x - s , y - t ) & ( 3 )\end{matrix}$$

Here, Ω represents the domain of integration of (σ, τ) (partial domain of R×R) and the second equation is a formula which assumes that the domain of integration is a rectangular area and divides it into 2 L parts in each of the horizontal and vertical directions to make an approximation calculation.

A model expressed by one scale as in Equation (1) is called SSR and a model expressed by a plurality of scales is called MSR. MSR expressed by N scales is represented by Equation (5) if the reflected light component of the i-th SSR shown in Equation (4) is combined with weight W.

$$\begin{matrix}Equation ( 4 ) & \\ {R}_{S S R , i} ( x , y ) = log I ( x , y ) - log [ {F}_{i} ( x , y ) ⊗ I ( x , y ) ] & ( 4 ) \\ Equation ( 5 ) & \\ {R}_{M S R} ( x , y ) = {\sum }_{i = 1}^{n} {W}_{i} {R}_{S S R , i} ( x , y ) & ( 5 )\end{matrix}$$

Next, the detailed structure of the image correcting section 40 will be described referring to FIG. 6.

The scale 1 filter section 43 and scale 2 filter section 45 of the image correcting section 40 are arithmetic processing sections which deal with the convolution product in Equation (3). The reflected light extracting section 49 includes the scale 1 filter section 43, the scale 2 filter section 45, logarithmic transformation sections 46, 44, and 42 and extracts the reflected light component.

Specifically, output 431 of the scale 1 filter section 43 is logarithmically transformed by the logarithmic transformation section 44 and its difference from the image data 21 which has been logarithmically transformed by the logarithmic transformation section 42 is calculated (signal 442). Also, output 451 of the scale 2 filter section 45 is logarithmically transformed by the logarithmic transformation section 46 and its difference from the image data 21 which has been logarithmically transformed by the logarithmic transformation section 42 is calculated (signal 462).

In short, the signal 442 and signal 462 are the reflected light component information of SSR in Equation (4).

After the signal 442 and signal 462 are multiplied by weights W1 and W2 respectively, they are added. The result is adjusted by gain G as necessary to become the reflected light component (signal 463) of the image data 21. In short, the reflected light component (signal 463) is the reflected light information of MSR in Equation (5).

Due to the above structure of the reflected light extracting section 49 (enclosed by dotted line in the figure), the influence of the illumination light component in the image data 21 can be eliminated and the reflected light component can be extracted.

The image correcting section 40 further includes: an exponential transformation section 47 which returns the reflected light component (signal 463) from the logarithmic luminance space to a linear luminance space; and a skin reconstruction signal generating section 48 which generates a skin area color 481 to replace the actual skin color in the image by a fixed skin color.

Thus, the image correcting section 40 returns the reflected light component (signal 463) to the linear luminance space by the exponential transformation section 47 and reconstructs the skin area with the skin area color 481 to obtain the image data (unpacked image information 41) as a corrected form of the image data 21.

The blood flow information 32 (hue information 35) and skin area mark information 31 which the blood flow analyzing section 30 has obtained by analyzing the image data 21 are entered into the local pulse wave detecting sections 50 (50a, 50b, 50c) (see FIG. 1) provided for the forehead 1, right buccal surface 2a, and left buccal surface 2b to detect the pulse wave information of the skin areas.

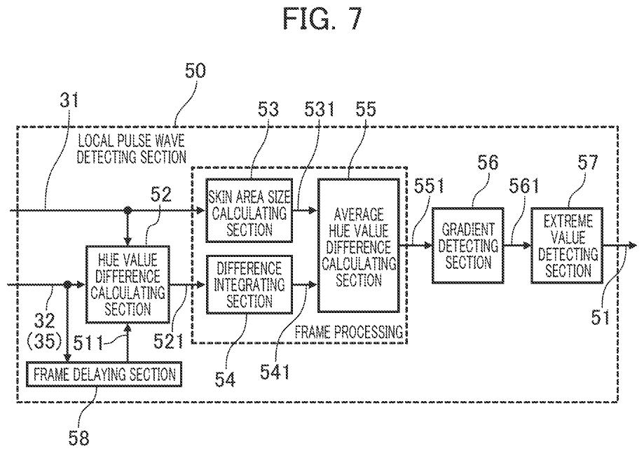

FIG. 7 is a structure diagram of the local pulse wave detecting section 50.

The local pulse wave detecting section 50 includes a frame delaying section 58, a hue value difference calculating section 52, a skin area size calculating section 53, a difference integrating section 54, an average hue value difference calculating section 55, a gradient detecting section 56, and an extreme value detecting section 57.

The frame delaying section 58 outputs delayed hue information 511 which is blood flow information 32 (hereinafter, hue information 35) time-delayed for one frame.

The hue value difference calculating section 52 receives the skin area mark information 31, hue information 35, and delayed hue information 511 and outputs hue difference information 521 which is set as follows according to “1” or “0” as the value of the skin area mark information 31.

If the hue value difference calculating section 52 receives the signal of a pixel in the skin area (namely, if 1 is entered as the skin area mark information 31), outputs the hue difference information 521 as the difference between the received hue information 35 and delayed hue information 511 (namely the difference between the hue information 35 of a frame and the hue information 35 of a frame preceding that frame). If the hue value difference calculating section 52 receives the signal of a pixel outside the skin area (namely, if 0 is entered as the skin area mark information 31), it outputs the color difference information 521 as value 0.

The skin area size calculating section 53 receives the skin area mark information 31 which indicates the inclusion in the skin area, and counts the number of pixels in the skin area of the frame to be processed (area for which the skin area mark information 31 is “1”) and outputs the count value as the skin area size information 531.

The difference integrating section 54 receives the hue difference information 521, integrates the values of the hue difference information 521 for the pixels in the skin area of the frame concerned and outputs the integrated value as integrated hue difference information 541.

The average hue value difference calculating section 55 receives the skin area size information 531 and integrated hue difference information 541 and outputs the value obtained by dividing the value of the integrated hue difference information 541 by the value of the skin area size information 531, as pulse wave information 551 for each frame. This pulse wave information 551 can be considered to be the amount of change in the average value of the hue difference information 521 of the pixels included in the skin area of the frame, namely the amount of change in the average value of the hue information 35 of the skin area of the living body (subject).

The gradient detecting section 56 is notified of the pulse wave information 551 for each frame.

The gradient detecting section 56 seeks the amount of temporal change in the pulse wave information 551 (namely, gradient). Then, it outputs the sign of the gradient as gradient information 561.

Since the pulse wave information 551 is a temporally differentiated form of the hue information 35, the gradient information 61 is the second order differential quantity of the hue information 35, which shows the gradient of the curve indicating the hue information 35

The extreme value detecting section 57 receives the gradient information 561 and seeks a frame for which the sign of the gradient has changed from a positive value to a negative value or a frame for which the sign of the gradient has changed from a negative value to a positive value. This means that at the time corresponding to the frame thus sought, the pulse wave information 551 has changed from increase to decrease or from decrease to increase, namely becomes the maximum or minimum value.

The extreme value detecting section 57 adds “1” as extreme value information to the pulse wave information 551 for a frame for which the sign of the gradient has changed from a positive value to a negative value and outputs it as the pulse information 51. For a frame for which the sign of the gradient has changed from a negative value to a positive value, it adds “−1” as extreme value information and for a frame for which the sign of the gradient has not changed, it adds “0” as extreme value information.

The pulse rate can be calculated from the interval of frames (number of frames) whose extreme value information of the pulse information 51 is “1” or “−1”.

The pulse wave velocity calculating section 60 acquires the pulse information 51 from each of the local pulse wave detecting section 50a for the forehead 1, the local pulse wave detecting section 50b for the right buccal surface 2a, and the local pulse wave detecting section 50c for the left buccal surface 2b. Then, among these pieces of pulse information 51, the time difference (number of frames) of frames whose extreme value information is “1” or “−1” is calculated and taken as pulse wave phase difference among the forehead 1, right buccal surface 2a, and left buccal surface 2b.

The pulse wave velocity calculating section 60 calculates the pulse wave velocity by dividing the difference in the distance from the heart between the areas subjected to pulse wave detection, by the pulse wave phase difference.

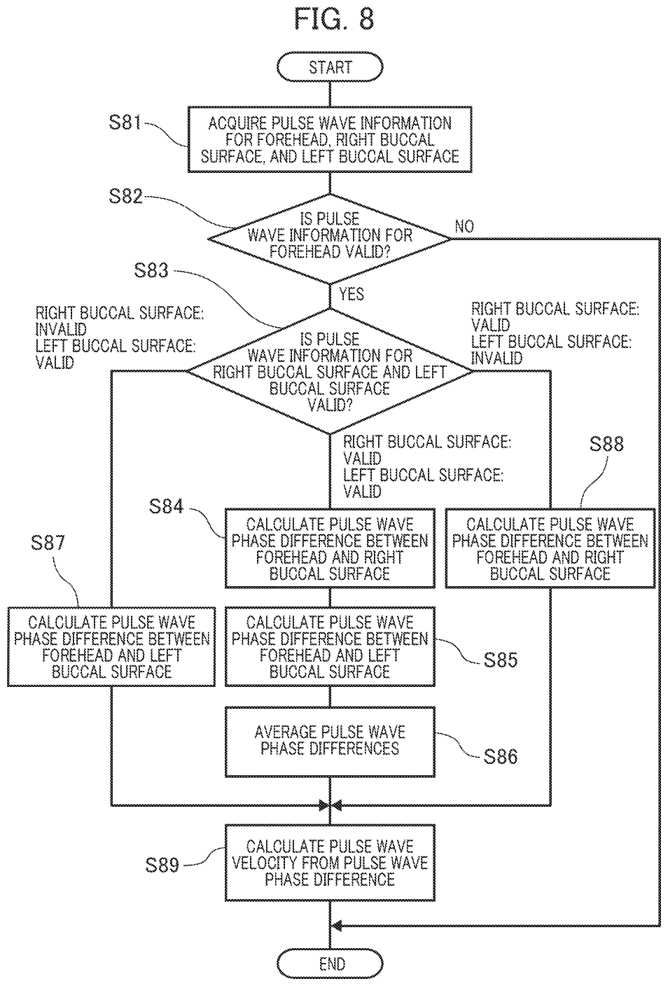

Next, the sequence in which the pulse wave velocity calculating section 60 acquires the pulse wave velocity will be explained in detail referring to FIG. 8.

At Step S81, the pulse wave velocity calculating section 60 acquires the pulse information 51 detected by the local pulse wave detecting sections 50 in the respective skin areas of the forehead 1, right buccal surface 2a, and left buccal surface 2b.

At Step S82, the pulse wave velocity calculating section 60 decides whether the acquired pulse information 51 for the forehead 1 is valid or not. The decision is made according to whether the pulse information 51 includes extreme value information (sign of gradient change) or not.

If the pulse information 51 for the forehead 1 is invalid (No at S82), the pulse wave phase difference cannot be calculated and the sequence is ended. If the pulse information 51 for the forehead 1 is valid (Yes at S82), the sequence proceeds to Step S83.

At Step S83, the pulse wave velocity calculating section 60 decides whether the pulse information 51 for the right buccal surface 2a and that for the left buccal surface 2b which have been acquired at Step S81 are valid or not. The decision is made according to whether the pulse information 51 includes extreme value information (sign of gradient change) or not.

If the pulse information 51 for the right buccal surface 2a is valid and the pulse information 51 for the left buccal surface 2b is also valid, the sequence proceeds to Step S84. If the pulse information 51 for the right buccal surface 2a is invalid and the pulse information 51 for the left buccal surface 2b is valid, the sequence proceeds to Step S87. If the pulse information 51 for the right buccal surface 2a is valid and the pulse information 51 for the left buccal surface 2b is invalid, the sequence proceeds to Step S88.

At Step S84, the pulse wave velocity calculating section 60 calculates the pulse wave phase difference from the pulse information 51 for the forehead 1 and the pulse information 51 for the right buccal surface 2a and the sequence proceeds to Step S85.

At Step S85, the pulse wave velocity calculating section 60 calculates the pulse wave phase difference from the pulse information 51 for the forehead 1 and the pulse information 51 for the left buccal surface 2b and the sequence proceeds to Step S86.

At Step S86, the pulse wave velocity calculating section 60 averages the pulse wave phase difference calculated at Step S84 and the pulse wave phase difference calculated at Step S85. Then, the sequence proceeds to Step S89.

At Step S87, the pulse wave velocity calculating section 60 calculates the pulse wave phase difference from the pulse information 51 for the forehead 1 and the pulse information 51 for the left buccal surface 2b and the sequence proceeds to Step S89.

At Step S88, the pulse wave velocity calculating section 60 calculates the pulse wave phase difference from the pulse information 51 for the forehead 1 and the pulse information 51 for the right buccal surface 2a and the sequence proceeds to Step S89.

At Step S89, the pulse wave velocity calculating section 60 calculates the pulse wave velocity from the pulse wave phase difference calculated at Step S87, Step S86, or Step S88 and ends the sequence.

In the abovementioned flow of processing by the pulse wave velocity calculating section 60, even in the case of a pulse wave detection failure that the pulse information 51 of the right buccal surface 2a or left buccal surface 2b cannot be detected, the pulse wave velocity can be calculated according to the detected pulse information 51.

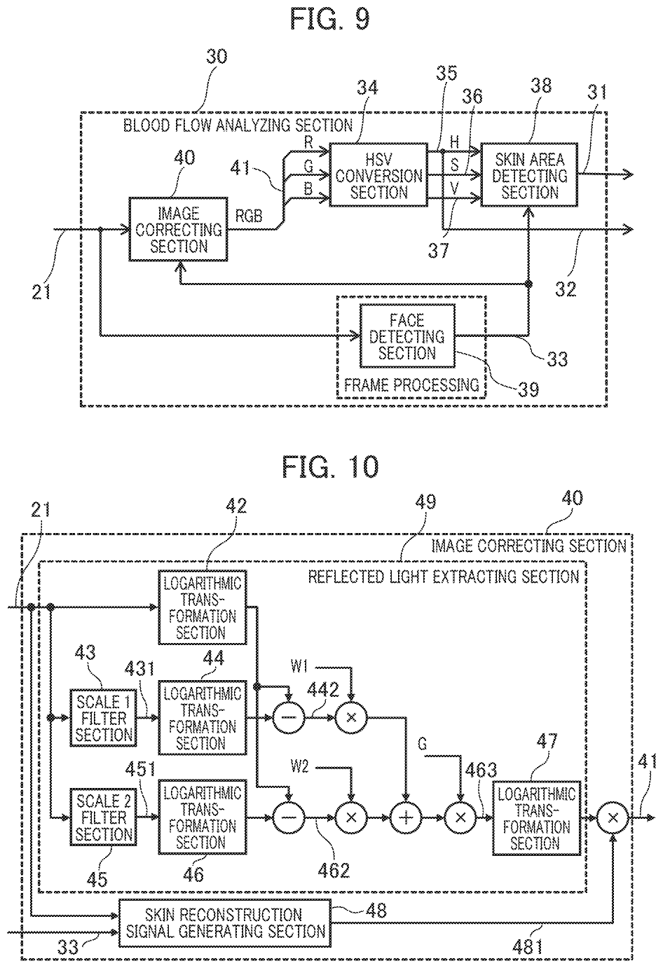

Next, another feature of the blood flow analyzing section 30 will be described referring to FIG. 9.

The blood flow analyzing section 30 in FIG. 9 is different from the blood flow analyzing section 30 in FIG. 5 in that the image correcting section 40 is notified of the face area mark information 33 which indicates the position information of the face area including the skin area for blood flow detection.

The other features are the same as in the blood flow analyzing section 30 illustrated in FIG. 5 and their description is omitted here.

FIG. 10 is a detailed structure diagram of the image correcting section 40 in FIG. 9, which receives the face area mark information 33.

The image correcting section 40 in FIG. 10 is different from the image correcting section 40 in FIG. 6 in that the skin reconstruction signal generating section 48 is notified of the face area mark information 33.

The other features are the same as in the image correcting section 40 illustrated in FIG. 6 and their description is omitted here.

Whereas the skin reconstruction signal generating section 48 in FIG. 6 outputs a fixed skin area color 481, the skin reconstruction signal generating section 48 in FIG. 10 receives the face area mark information 33 as an additional input signal and stores the skin color from the face area, for example, of a single frame or an average of two or more frames and outputs the stored signal for the skin area color 481. If the face area mark information 33 is non-signal (signal input is 0) or smaller than a previously specified threshold, it should be taken as a face detection failure and when a face area signal is received again, the skin color information of a single frame or an average of two or more frames may be stored.

According to the above feature, the face in an image can be identified and the skin area color change suitable for each individual person can be captured and pulse detection can be made appropriately.

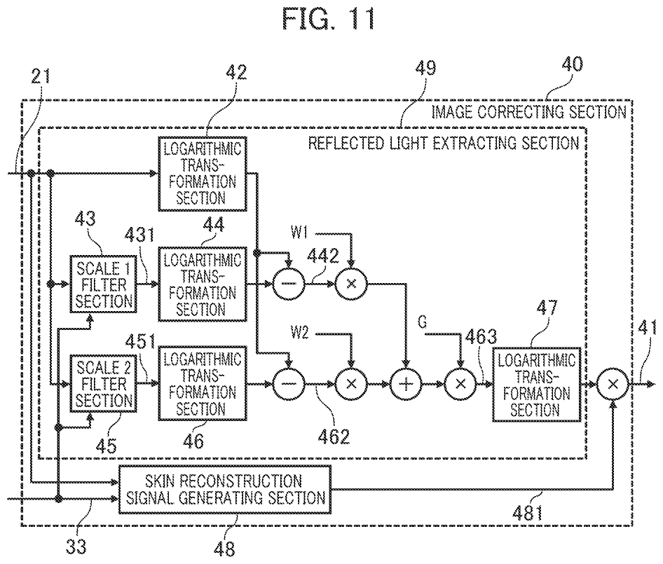

FIG. 11 explains a further feature of the image correcting section 40 in FIG. 9.

The image correcting section 40 in FIG. 11 is different from the image correcting section 40 in FIG. 10 in that the scale 1 filter section 43 and scale 2 filter section 45 are notified of the face area mark information 33, as an additional feature. The other features are the same as in the image correcting section 40 in FIG. 10 and their description is omitted here.

The scale 1 filter section 43 and scale 2 filter section 45 perform arithmetic operation of the convolution product in Equation (3) for the image data 21 of the face area including the skin area for blood flow detection which is indicated by the face area mark information 33.

As mentioned above, in the biological information detection device according to the embodiment, a pulse wave is detected according to the image data of a given skin area in the face area. Therefore, even when correction of the image data 21 is not made for an area other than the face area, the pulse wave detection accuracy is not affected. In the image correcting section 40 in FIG. 11, the amount of arithmetic operation by the scale 1 filter section 43 and scale 2 filter section 45 can be reduced and the processing load on the biological information detection device can be reduced.

The present invention is not limited to the above embodiment but includes many variations. The above embodiment has been described in detail for easy understanding of the present invention. However, the present invention is not limited to a structure which includes all the elements described above. An element of an embodiment may be replaced by an element of another embodiment or an element of an embodiment may be added to another embodiment.

What is claimed is:

1. A biological information detection device comprising:
an image acquiring section that acquires image information by taking an image of a face of a living body;
a blood flow analyzing section that corrects the image information according to a Retinex theory for color constancy, outputs hue information in the corrected image information as blood flow information, and outputs skin area mark information indicating a position of a given skin area in the face; and
a local pulse wave detecting section that obtains, from the blood flow information in the skin area corresponding to the skin area mark information, pulse information of the skin area.

2. The biological information detection device according to claim 1,
wherein the blood flow analyzing section corrects the image information using the image information and output of a plurality of filter sections that generate a convolution product of Gaussian distributions with different scales and the image information.

3. The biological information detection device according to claim 2,
wherein the blood flow analyzing section includes a face detecting section that outputs face area mark information indicating a position of a face area in the image information, and
wherein the filter sections perform filtering of an area indicated by the face area mark information in the image information.

4. The biological information detection device according to claim 1, wherein the corrected image information is reconstructed with fixed skin area color.

5. The biological information detection device according to claim 1, wherein the corrected image information is reconstructed with color of a face area indicated by face area mark information in the image information.

6. The biological information detection device according to claim 1, comprising:
the local pulse wave detecting section being provided in plurality, and
the device further comprising:
a pulse wave velocity calculating section that calculates pulse wave velocity from phase difference of pulse information of the plural skin areas as calculated by the plural local pulse wave detecting sections; and
a blood pressure estimating section that estimates blood pressure according to the pulse wave velocity.

7. The biological information detection device according to claim 6,
wherein the blood flow analyzing section analyzes image data of at least three skin areas including a first skin area lying on a centerline of the face and a pair of second skin areas lying symmetrically with respect to the centerline with a blood flow path nearer to a heart than the first skin area, to obtain blood flow information, and
wherein the pulse wave velocity calculating section calculates pulse wave velocity from phase difference between pulse information of the first skin area and pulse information of one of the second skin areas.
